(19) United States
(12) Patent Application Publication
(10) Pub. No.: US 2014/0073591 A1
(43) Pub. Date: Mar. 13, 2014

(54) MDR METHOD AND PRODUCTS FOR TREATING MRSA

(71) Applicants: Wayne Michael Putnam, Lexington, KY (US); Beth D. Gudeman, Lexington, KY (US)

(72) Inventors: Beth D. Gudeman, Lexington, KY (US); Wayne Michael Putnam, Lexington, KY (US)

(73) Assignee: CBA Pharma, Inc., Lexington, KY (US)

(21) Appl. No.: 14/025,148

(22) Filed: Sep. 12, 2013

Related U.S. Application Data
(60) Provisional application No. 61/700,708, filed on Sep. 13, 2012.

(51) Int. Cl. A61K 31/4725; A61K 31/431; A61K 31/546; A61K 31/5383; A61K 31/43; A61K 31/7052

(52) U.S. Cl. CPC A61K 31/4725; A61K 31/43; A61K 31/7052; A61K 31/546; A61K 31/5383; A61K 31/431
USPC 514/29; 514/308; 514/198; 514/206; 514/230.2; 514/197; 514/196

(57) ABSTRACT

Multidrug resistance reversers of the d-tetrandrine family are used in conjunction with penicillins and other principle drugs used to treat MRSA.

### FIELD AND BACKGROUND OF THE INVENTION

The present invention relates to the treatment of methicillin-resistant Staphylococcus aureus (MRSA). Methicillin has been used to treat infections caused by GRAM-positive bacteria, especially beta-lactamase producing organisms such as Staphylococcus aureus, that are resistant to most penicillins. It has been largely replaced by flucloxacillin and dicloxacillin. However the term methicillin-resistant Staphylococcus aureus is used to describe Staphylococcus aureus strains which are resistant to all penicillins. The resistance of MRSA to a wide range of antibiotics results in MRSA also being referred to as “multidrug-resistant Staphylococcus aureus.”

Penicillins such as methicillin, oxicillin, flucloxacillin and dicloxacillin are “beta-lactam antibiotics.” They act by inhibiting the synthesis of bacterial cell walls. It is believed that the resistance of MRSA is based on its production of beta-lactamase, which digests the beta-lactam provided by the beta-lactam antibiotics. This contrasts multi-resistance pump mechanism responsible for multidrug resistance in most types of cancer and multidrug resistant disease.

### SUMMARY OF THE INVENTION

In the present invention, multidrug resistance reversers of the d-tetrandrine family are used in conjunction with penicillins and other principle drugs used to treat MRSA.

### DESCRIPTION OF THE PREFERRED EMBODIMENT

The d-tetrandrine family members have been found effective in treating multi-drug resistance in a variety of diseases and conditions, including cancer and malaria. See U.S. Pat. Nos. 5,025,020; 5,332,747; 6,528,519; 6,911,454; 6,124,315 and 6,967,927. The d-tetrandrine family members have the following structural formula:

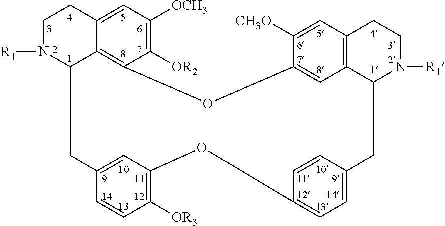

where R₁ and R₁′ are the same or different shortchained carbon based ligand including without limitation, CH₃, CO₂CH₃ or H; and R₂ is CH₃ or C₂H₅; and R₃ is CH₃ or hydrogen; and where the chemical structure has the “S” isomeric configuration at the C-1′ chiral carbon location.

The preferred members of the d-tetrandrine family include the following representative examples, which are no intended to be exhaustive: d-tetrandrine, isotetrandrine, hernandezine, berbamine, pycnamine, phaeanthine, obamegine, ethyl fangehinoline and fangchinoline. In all of these examples, R₁ and R₁′ constitute the methyl group. Variation within the group occurs in that R₂ and R₃ may constitute either a methyl group or hydrogen, and the isometric configuration of the compounds at the C-1 and C-1′ chiral carbon positions is either R (rectus) or S (sinister). The rules for R and S configuration can be found in Morrison and Boyd, “Organic Chemistry,” ^{4th} Edition, copyright 1983 by Allyn and Bacon, at pp. 138-141, As Doted above, the chiral configuration at C-1′ is “S” for members of the d-tetrandrine family. In addition, hernandezine includes a methoxy group at the C-5 position.

The most preferred member of the claimed tetrandrine family is d-tetrandrine. Methods for extracting and/or purifying d-tetrandrine are disclosed in U.S. Pat. Nos. 6,218,541 and in Published Patent Application No. 2011/0105755.

The d-tetrandrine, family member is used in conjunction with an antibiotic, referred to herein as a “principle drug,” for treating MRSA. The most preferred principle drugs include the antibiotics ampicillin (AMP), azithromycin (AZM), cefazolin (CFZ) and levofloxacin (LEV).

In addition, the drugs to which MRSA is normally resistant may be used as the principle drug, in combination with the d-tetrandrine family member. These include the penicillins (methicillin, deloxacillin, nafcillin, oxacillin, et and the cephalosporins. These principle drugs are used at Their normal dosage levels, though some reduction in the amounts administered may be possible, given the effect of the d-tetrandrine family member.

The d-tetrandrine family member and the principle MRSA drug can be formulated together into a single formula, they can be formulated separately and administered either simultaneously or sufficiently close together that the MRSA is exposed to both simultaneously. The two drugs formulated separately may be sold as part of a “kit.” The usage ratio of the d-tetrandrine family member to a principle MRSA drug, will vary from patient to patient and as a function of the principle MRSA drug used, within a range of from about 0.04 to about 170, more typically from about 1 to 100.

It is believed that the optimum dosage procedure would be to administer the d-tetrandrine family multidrug resistance reverser in oral doses of from about 50 to about 1000 mg per square meter per day, more preferably 250-700, and most preferably about 500, (probably in two to four doses per day) over a period of from about 4 to about 14 days. The dosage level for the d-tetrandrine family member will vary from case to case, based on the patient and on the primary MRSA drug used. The primary MRSA drug is then administered at usual dosage levels (possibly somewhat less in view of the potentiation effect of the resistance reverser) once or more during the course of the resistance reverser dosing. For example, during a four day period of d-tetrandrine administration, the primary MRSA drug would be administered on the beginning of the third day. Over a 14 day period, the primary MRSA drug, or drugs might be administered on day 5 and day 10, or on days 4, 8 and 12.

The d-tetrandrine family bisbenzylisoquinolines have two nitrogen locations and hence can exist in the free base form or as a mono or di-acid salt. Because of the enhanced solubility of the salt form of pharmaceutical ingredients, the salt forms are used in formulating pharmaceutical compositions. The active ingredient thus solubilizes more quickly and enters the bloodstream faster. The free base form is not soluble in water. However, it has recently been surprisingly found by a co-worker that the free base formulations of d-tetrrodrine family members are absorbed into the bloodstream substantially as rapidly as formulations of the di-acid salt members of the family. Accordingly, we propose to use either the free base or the di-acid salt of the d-tetrandrine family member in our MRSA MDR formulations.

The preferred formulations comprise a member of the d-tetrandrine family combined with a suitable pharmaceutical carrier. The pharmaceutical carrier can be a liquid or a solid composition. A liquid carrier will preferably comprise water, possibly with additional ingredients such as 0.25% carboxymethylcellulose. The solid carrier or diluent used may be pregelatinized starch, microcrystalline cellulose or the like. It may also be formulated with other ingredients, such as colloidal silicone dioxide, sodium lauryl sulfate and magnesium stearate.

A 200 mg capsule, tablet or liquid dosage formulation is most preferred. The most preferred dose of about 500 mg/square meter/day is roughly 1000 tug per day for a 190 pound patient six feet tall. Such a patient can fulfill the dosage requirements by taking five capsules during the course of the day, for example three in the morning and two in the evening, or one at a time spaced out over the day. A smaller person weighing 125 pounds at a height of five feet six inches would require four 200 mg capsules during the course of the day.

Of course, it is understood that the above disclose some embodiments of the invention, and that various changes and alterations can be made without departing from the scope of the invention as set forth in the attached claims and equivalents thereof.

1. A method of treating MRSA comprising
administering to a patient affected with MRSA a member of the d-tetrandrine family having the following structural formula: 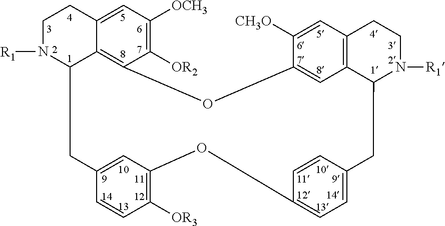
where R₁ and R₁′ are the same or different short chained carbon based ligand including without limitation, CH₃, CO₂CH₃ or H; and R₂ is CH₃ or C₂H₅; and R₃ is CH₃ or hydrogen, has the “S” isomeric configuration at the C-1′ chiral carbon location.

2. The method of claim 1 wherein said member of the d-tetrandrine family is d-tetrandrine.

3. The method of claim 1 in which the d-tetrandrine family member is used in conjunction with a principle drug for treating MRSA.

4. The method of claim 3 in which the principle drugs administered includes one or more of the following antibiotics: ampicillin (AMP), azithromycin (AZM), cefazolin (CFZ) and levofloxacin (LEV).

5. The method of claim 3 in which the principle drugs administered comprises drugs to which MRSA is normally resistant.

6. The method of claim 3 in which the principle drugs administered includes one or more of the penicillins.

7. The method of claim 3 in which the principle drugs administered includes one or more of the following antibiotics: methicillin, dicloxacillin, nafcillin, and oxacillin.

8. The method of claim 3 in which the principle, drugs administered includes one or more of the cephalosporins.

9. The method of claim 3 in which the d-tetrandrine family member and the principle MRSA drug are formulated together into a single formula.

10. The method of claim 3 in which the d-tetrandrine family member and the principle MRSA drug are formulated separately and administered either simultaneously or sufficiently close together that the MRSA is exposed to both simultaneously.

11. The method of claim 3 in which the d-tetrandrine family member and principle MRSA drug are administered in a usage ratio of d-tetrandrine family member to principle MRSA drug, within a range of from about 0.04 to about 170.

12. The method of claim 3 in which the d-tetrandrine family member and principle MRSA drug are administered in a usage ratio of d-tetrandrine member to principle MRSA drug, within a range of from about 1 to 100.

13. The method of claim 3 in which the d-tetrandrine family is administered in oral doses of from about 50 to about 1000 mg per square meter per day over a period of from about 4 to about 14 days, and the primary MRSA drug is then administered at usual dosage levels once or more during said 4 to 14 days.

14. The method of claim 13 in which the d-tetrandrine family is administered in oral doses of from about 250-700 mg per square meter per day over said period of from about 4 to about 14 days.

15. The method of claim 13 in which the d-tetrandrine family is administered in oral doses of about 500 mg per square metes per day over said period of from about 4 to about 14 days, in two to four doses per day.

16. A pharmaceutical composition comprising a principle drug for treating MRSA, combined with a member of the d-tetrandrine family having, the following structural formula: 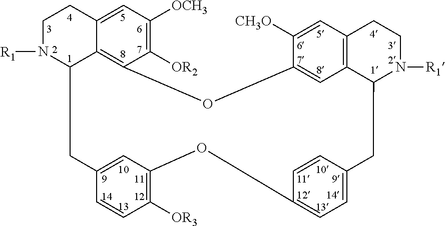
where R₁ and R₁′ are the same or different short chained carbon based ligand including without limitation, CH₃, CO₂CH₃ or H; and R₂ is CH₃ or C₂H₅; and R₃ is CH₃ or hydrogen, has the “S” isomeric configuration at the C-1′ chiral carbon location.

17. A pharmaceutical kit including a principle drug for treating MRSA, and a formulation comprising a member of the d-tetrandrine family having the following structural formula: 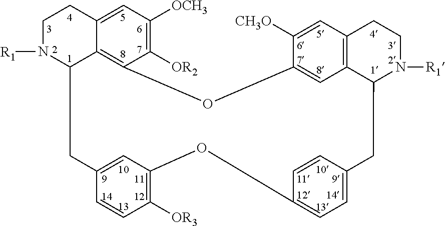
where R₁ and R₁′ are the same or different short chained carbon based ligand including without limitation CH₃, CO₂CH₃ or H; and R₂ is CH₃ or C₂H₅; and R₃ is CH₃ or hydrogen, has the “S” isomeric configuration at the C-1′ chiral carbon location.
